(19) United States
(12) Patent Application Publication
(10) Pub. No.: US 2015/0126868 A1
(43) Pub. Date: May 7, 2015

(54) PIERCING-LIKE REFILLABLE DRUG FEEDING DEVICE

(71) Applicant: Adrian SCHMIDT, Deisenhofen (DE)

(72) Inventor: Adrian Schmidt, Deisenhofen (DE)

(21) Appl. No.: 14/394,283

(22) PCT Filed: Apr. 19, 2013
(86) PCT No.: PCT/EP2013/001170

§ 371 Date: Oct. 14, 2014

(30) Foreign Application Priority Data
Apr. 24, 2012 (EP) 12002873.3

(51) Int. Cl. A61M 5/32; A61M 5/31

(52) U.S. Cl. CPC A61M 5/329; A61M 5/31; A61M 2210/167
USPC 600/439; 604/264; 604/256; 604/244; 604/506

(57) ABSTRACT

A device for delivering a drug comprising an elongate hollow body having a first longitudinal end and a second longitudinal end. The hollow body includes a radially outer peripheral wall, which surrounds a radially inner, and elongate hollow space. The peripheral wall of the hollow body has at least one drug outlet opening between the two longitudinal ends.

### CROSS-REFERENCE TO RELATED APPLICATIONS

This application is a U.S. National Phase application under 35 U.S.C. §371 of International Application No. PCT/EP2013/001170, filed on Apr. 19, 2013, and claims benefit to European Patent Application No. EP 12002873.3, filed on Apr. 24, 2012. The International application was published in German on Oct. 31, 2013 as WO 2013/159889 A1 under PCT Article 21(2).

### FIELD

The invention relates to a device for delivering a drug, in particular into the tissue of a living human or animal, comprising an elongate hollow body having a first longitudinal end and a second longitudinal end, wherein the hollow body comprises a radially outer peripheral wall, which surrounds a radially inner, elongate hollow space.

### BACKGROUND

A number of devices for delivering a drug into the body of a living human or animal are already known in medicine. These are generally devices whose hollow body is of tube-like form, the hollow space extending continuously from the first to the second longitudinal end of the hollow body. Such hollow bodies are designed, for example, as a hollow needle, also called a “cannula”, and are generally used to inject a drug into the tissue to be treated or directly into the bloodstream of the human or animal by means of a syringe comprising a hollow cylinder and a plunger. So-called “catheters” are a further example. These are small tubes or hoses of different diameters, with which hollow organs, such as the bladder, vessels, etc., can be probed, emptied, filled or flushed.

For appropriate use of these drug delivery devices, it is generally necessary to puncture, i.e. pierce, the skin of the human or animal to be treated, so that the drug can be guided from an extracorporeal source, via an extracorporeally arranged longitudinal end of the hollow body and via the elongate hollow space, to an outlet opening at an intracorporeally arranged longitudinal end of the hollow body. The puncturing is sensed by many people to be painful or at least unpleasant, and some people have developed downright phobias of syringes. This problem is of special importance particularly for people who have to be supplied with a drug repeatedly at more or less regular intervals, such as for example diabetics who are regularly dependent on the supply of insulin from an extracorporeal source.

There are also catheters which are suitable for a prolonged duration in situ in the body, where a prolonged duration in situ is to be understood as a duration in situ of several days or even longer, however, such catheters in practice have various disadvantages. In particular, permanently secure fixing without restricting the freedom of movement of the person concerned is frequently an unsolved problem. Sometimes, the elongate hollow body of such a catheter is firmly bonded to the skin around the puncture site using a suitable adhesive. However, there is the risk here that germs may remain under the bonding site and proliferate there. This may, in turn, lead to serious infections if the germs then penetrate into the body via the puncture site.

A special field of application for drug delivery devices with which the present invention is particularly concerned relates to the treatment of erectile dysfunction in men. Although there are impotence drugs, such as e.g. sildenafil, better known under the trade name Viagra® of the US company Pfizer, which can be taken orally to cause an erection, there are people for whom such orally taken impotence drugs—for whatever reasons—do not achieve the desired effect or else cause undesired side effects. These people then frequently have to rely on injecting an appropriate drug directly into the penis, in particular directly into at least one corpus cavernosum of the same.

In addition to the frequently felt discomfort with puncturing in this sensitive area of the body, further problems may arise in this special field of application. Generally, the puncturing is performed shortly before sexual intercourse by the patient himself, i.e. without the help of medically trained staff There is thus an increased risk of the outlet opening of the hollow needle not being correctly positioned and of the drug not reaching the corpus cavernosum or not in sufficient quantity, so that the desired erection does not occur. In this case, the unpleasant puncturing has to be performed again. Problems may also arise with regard to the sterility of the puncture site or of the hollow needle used, if the puncturing is done without the help of medically trained staff. Furthermore, there is a risk of the frequently very thin hollow needle breaking off during the puncturing in the region of the penis, so that in some cases it can only be removed by means of a surgical procedure.

### SUMMARY

In an embodiment, the present invention provides a device for delivering a drug. The device comprises an elongate hollow body having a first longitudinal end and a second longitudinal end. The hollow body includes a radially outer peripheral wall, which surrounds a radially inner, and elongate hollow space. The peripheral wall of the hollow body has at least one drug outlet opening between the two longitudinal ends.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a cross-sectional view through a penis, through which a drug delivery device according to the invention is passed;

FIG. 2a shows a cross-sectional view of a first embodiment of a closure arrangement arranged at a longitudinal end of the hollow body;

FIG. 2b shows a cross-sectional view of a variant of the first embodiment according to FIG. 2a, wherein the variant comprises a receptacle for a drug reservoir and a sharp-edged projection for targeted destruction of the drug reservoir;

FIG. 3 shows a cross-sectional view of a second embodiment of a closure arrangement arranged at a longitudinal end of the hollow body;

FIG. 4 shows a cross-sectional view of a third embodiment of a closure arrangement arranged at a longitudinal end of the hollow body;

FIG. 5 shows a cross-sectional view of a fourth embodiment of a closure arrangement arranged at a longitudinal end of the hollow body;

FIG. 6 shows a cross-sectional view of a fifth embodiment of a closure arrangement arranged at a longitudinal end of the hollow body; and

FIG. 7 shows a cross-sectional view of a section of the hollow body of the drug delivery device according to the invention, which section has a drug outlet opening.

### DETAILED DESCRIPTION

An aspect of the present invention is to improve devices for delivering a drug such that the above-mentioned problems can be, at least partly, avoided.

In an embodiment, this aspect can be achieved according to the invention by a device for delivering a drug, in which the peripheral wall of the hollow body has at least one drug outlet opening between the two longitudinal ends. The at least one drug outlet opening allows the delivery device to be positioned in such a manner in the body of the human or animal to be treated that both the first longitudinal end and the second longitudinal end of the hollow body are arranged extracorporeally, whereas a section of the hollow body arranged between these two longitudinal ends, which has the at least one drug outlet opening, is arranged intracorporeally. It is thereby furthermore possible, for example by attaching an element to the longitudinal ends of the hollow body which has a broad diameter than the hollow body, to reliably permanently prevent the hollow body from unintentionally slipping out of the human or animal body, even when the body moves. Thus, the delivery device can remain, at least in sections, permanently in the body without problems. It is not necessary for a puncturing of the skin to be performed again and again. Rather, the drug can be delivered to the human or animal body repeatedly via the delivery device. Also a material failure of the delivery device at an intracorporeal location is largely unproblematic in the case of the delivery device according to the invention, since the two longitudinal ends are positioned outside the body and can thus be withdrawn individually in different directions in the event of rupture of the hollow body.

The at least one drug outlet opening passes through the peripheral wall of the hollow body between the two longitudinal ends of the hollow body preferably in the radial direction. The at least one drug outlet opening can be formed, for example, as a substantially circular or oval or slit-shaped through-hole in the peripheral wall of the hollow body of the delivery device. Thereby, the hollow body can have substantially the shape of a straight circular hollow cylinder. This is particularly advantageous when the hollow cylinder is produced from a metal or another relatively solid material which is to be passed rectilinearly through the corresponding body part, e.g. the penis. It is, however, also possible for the hollow body to have a different shape. For example, it may be produced from a relatively flexible, elastic material, such as a suitably soft plastic, so that it is elastically deformable.

Unlike the hollow needles and catheters known from the prior art, it is not mandatory in the case of the delivery device according to the invention for the hollow space to extend continuously from the first to the second longitudinal end of the hollow body. In principle, it is sufficient if the elongate hollow space, starting from one of the two longitudinal ends of the hollow body, extends into the hollow body but ends before reaching the opposite longitudinal end of the hollow body, similar to a blind hole. In this way, the second longitudinal end of the hollow body can be designed accordingly more stable, e.g. as a solid profile.

Nevertheless, it is, however, generally preferable if the delivery device according to the invention is of tube-like form, wherein the hollow space extends continuously from the first to the second longitudinal end of the hollow body, similar to the cannulas and catheters already known. In this way, the drug can be delivered to the human or animal body selectively via the first longitudinal end or the second longitudinal end of the hollow body. This affords an increased functional reliability by redundancy, since operatability of the delivery device according to the invention is still ensured even if the hollow space should become blocked over time in the region of one of the two longitudinal ends of the hollow body. Furthermore, this configuration affords the advantage that, for the positioning of the hollow body in the human or animal tissue, a piercing needle can be used which is passed through the lumen of the hollow body, and on piercing the human or animal tissue, e.g. the penis, takes along the hollow body with it and guides it through the tissue. Thereby, the piercing needle can at the same time impart stability to the hollow body during the positioning operation, which is particularly important if the hollow body is produced from a relatively soft material, for example a flexible plastic.

Likewise for reasons of redundancy, which leads to longer-lasting operatability of the delivery device according to the invention, it is proposed that the hollow body has a plurality of drug outlet openings. This results additionally in better distribution of the drug in the human or animal tissue, so that the desired effect can be achieved more quickly and efficiently. In particular for the use of the delivery device for treating erectile dysfunction, it is advantageous if the hollow body has at least two drug outlet openings, namely one for each of the two corpora cavernosa. It is even more preferable, however, if the hollow body has four drug outlet openings, namely two for each corpus cavernosum, since in this case there is redundancy for each corpus cavernosum. In principle, it is also conceivable to provide more than four drug outlet openings on the hollow body, but it must be taken into consideration that each drug outlet opening reduces a little the structural stability of the peripheral wall of the hollow body.

If the hollow body has a plurality of drug outlet openings, it is proposed in a continuation of the invention to arrange the drug outlet openings spaced apart from one another with respect to the direction of the longitudinal extent of the hollow body, in order not to cause excessive structural weakening of the peripheral wall of the hollow body locally.

In order to prevent bacteria or other pathogens from penetrating into the human or animal body via the drug delivery device according to the invention, and in order to prevent a drug introduced at one end of the hollow body from escaping unintentionally at the other end of the hollow body, it is proposed that the delivery device furthermore comprises at least one closure arrangement, preferably two closure arrangements, which is or are attached and/or attachable to a respective longitudinal end of the hollow body, in order to sealingly close the hollow space at the respective longitudinal end of the hollow body.

For the delivery of the drug and thus for the ensuring of the functionality of the delivery device, the at least one closure arrangement should, however, be suitable for enabling the delivery of a drug from outside the delivery device into the hollow space. In order to guarantee this, it may in some cases be necessary to dispense with the aforementioned sealing for preventing penetration of bacteria or other pathogens for a short time.

For example, it may be envisaged that at least one of the at least one closure arrangement is detachably connected or connectable, in particular by means of a screwed connection, to the hollow body. “Detachably” in this context means that the connection can be made and broken again repeatedly without destroying a material cohesion, which can only be restored with auxiliary means. For example, one longitudinal end of the hollow body can be provided with an external-thread section, whereas a closure arrangement assigned to this longitudinal end of the hollow body is provided with a complementarily formed internal-thread section. In this case, the said closure arrangement can in a simple manner be repeatedly screwed onto the hollow body and removed therefrom again for delivery of the drug.

Alternatively, it is also possible for at least one, preferably both, of the at least one closure arrangement to be non-detachably connected, in particular by means of an adhesive bonded connection, to the hollow body. “Non-detachably” in this context means that to disconnect the closure arrangement from the hollow body, a material cohesion must be irreversibly severed. The latter can only be restored by auxiliary means, such as e.g. reapplication of an adhesive agent. Since, however, it is generally impractical to connect the closure arrangement to the hollow body again using auxiliary means after each appropriate use of the drug delivery device according to the invention, it is proposed, particularly in those cases in which the closure arrangement is non-detachably connected to the hollow body, that the corresponding closure arrangement is suitable for allowing the delivery of a drug, even if the closure arrangement remains connected to the hollow body.

For example, at least one of the at least one closure arrangement can comprise a closure means suitable for opening and reclosing. This closure means can be designed, for example, in the form of a closure cap which is either formed integrally with the rest of the closure arrangement, e.g. by means of a joint, in particular a hinge joint, in particular a film hinge joint, or as a separate component therefrom. For example, a section of the closure arrangement can comprise an external thread and a separately formed closure cap can comprise a complementarily formed internal-thread section, similar to the valve closure of a bicycle tyre.

An embodiment which is particularly easy to use and therefore particularly preferred provides that at least one of the at least one closure arrangement comprises an elastic body, for example plug, which is suitable for being pierced with a pointed needle, in order to introduce a drug into the interior of the hollow body, and which is suitable, on account of its elastic properties, for sealingly reclosing the hollow space at the corresponding longitudinal end of the hollow body when the needle is withdrawn. By using closure arrangements formed in this manner, it is possible to avoid, for example, screwing off and on a component, which is frequently delicately formed and which otherwise may involve appropriate effort for the person concerned, while at the same time the risk that the screwed-off component may be lost is reduced. The closure arrangement having a plug can be formed, for example, as a “rubber port system” known in medical engineering. In particular, the elastic body can, at least in sections, be surrounded by a metallic ring which gives it structural support and affords the injection needle a certain guidance. Thereby, it is particularly advantageous if the metallic ring is funnel-shaped and tapers towards the hollow space of the hollow body, in order to guide the tip of the injection needle, with which the drug can be introduced into the hollow space of the delivery device, in the direction towards the hollow space of the delivery device. Thus, a surface area for placing the injection needle, which is available to the user of the delivery device according to the invention can be significantly greater than the cross-sectional area orthogonal to the direction of the longitudinal extent of the elongate hollow-body hollow space.

It should be noted at this point that embodiments are also conceivable, in which a syringe with injection needle is not necessary to introduce the drug into the hollow space of the delivery device according to the invention. For example, there could be provided on at least one closure arrangement a connection piece which can be coupled to a correspondingly complementarily formed counter-connection piece of an applicator containing the drug, for example in the manner of a bayonet fastening. Furthermore, it would be conceivable to use a pipette, instead of a syringe with an injection needle, in similar fashion to taking nose drops, in particular if the delivery device according to the invention has a funnel formed correspondingly large for receiving the end of the pipette.

It would also be conceivable for the delivery device according to the invention to comprise a pointed or sharp-edged projection which is preferably concealed as long as the access to the interior of the hollow body is closed by a closure arrangement at one of the longitudinal ends of the hollow body. Such a projection could be used to destroy an envelope of a suitably formed drug reservoir in a targeted local manner, so that an amount, preferably a precisely predetermined amount, of drug can be released from the reservoir into the interior of the hollow body.

An advantageous continuation of this concept provides for at least one drug reservoir, or a receptacle for a drug reservoir, respectively, to be provided in or at the delivery device according to the invention itself, preferably in or at one of the closure arrangements. For example, a closure arrangement could comprise or receive a rubber sphere or the like serving as a drug reservoir, or else could itself be formed substantially out of such a rubber sphere. The rubber sphere or the like can then be pressed against a pointed or sharp-edged projection of the device, for example by a firm pressure, in order to release a predetermined amount of drug in a targeted manner into the interior of the hollow body. In this way, a man who uses the delivery device according to the invention in the case of virility problems in the region of his penis can apply the drug discreetly and in an uncomplicated manner, as required, without having to handle a syringe and injection needle. The replacement of the destroyed drug reservoir with a new, intact drug reservoir can then be done at a later time in preparation for the next sexual act. In particular, it could be conceived to provide both longitudinal ends of the hollow body each with one receptacle for a corresponding drug reservoir in the closure arrangements, so that the device can be used in accordance with its normal operation also twice in succession, as required, without having to be recharged in the meantime. The closure arrangements are in this case to be designed such that they enable, preferably by the targeted application of pressure, the drug reservoir to be reliably opened, for example by bursting it. It would also be possible for the drug reservoirs to have a predetermined breaking point which is formed in such a manner that the drug reservoirs open, solely by applying an appropriately determined pressure without the necessity to provide a pointed or sharp-edged projection at the delivery device according to the invention for this purpose, against which the drug reservoir is to be pressed.

If the delivery device according to the invention is used to deliver an impotence drug to a penis, for example alprostadil or prostaglandin E1 or a mixture comprising papaverine HCL, phentolamine mesylate and prostavasin, in rare cases an overreaction can occur in which the erect penis no longer detumesces within an acceptable time. This is, on the one hand, very unpleasant for the person concerned and can, on the other hand, also result in serious damage to the vessels if no countermeasures are taken in time. A possible countermeasure is in particular the delivery of a corresponding antidote. With regard to the continuation set out above, it would therefore be conceivable to provide a reservoir for an impotence drug at one longitudinal end of the hollow body of the delivery device according to the invention, and to provide a reservoir for a corresponding antidote at the other longitudinal end. The delivery device according to the invention can thus comprise a plurality of different drugs in reservoirs spatially separated from one another.

In order to permanently ensure secure holding of the delivery device according to the invention in the human or animal body, and in order to prevent the delivery device from unintentionally slipping out of the body, even when the body moves, it is proposed that at least one, preferably all, of the at least one closure arrangement extends or extend in the radial direction of the hollow body radially beyond the hollow body outwards, in particular beyond a section of the hollow body situated between the two longitudinal ends of the hollow body. In particular when both closure arrangements, each assigned to one longitudinal end of the hollow body, respectively, have an appropriate radial dimension, it can be reliably ensured that the delivery device cannot slip out of the pierced body, neither part in one nor in the other direction.

Furthermore, it is proposed that the peripheral wall has a minimal, or substantially constant over the entire length, outside diameter in the radial direction, which is ≧0.26 mm, preferably between 0.26 mm and 0.9 mm, for example 0.4 mm. Catheters and cannulas already known in medical engineering are generally standardised regarding their size, in particular regarding their outside diameter, whereby the size is frequently specified in gauge (G). G 20 corresponds to an outside diameter of 0.9 mm, G 27 to an outside diameter of 0.4 mm and G 33 to an outside diameter of 0.26 mm. On the one hand, the outside diameter of the hollow body to be passed through the relevant body part of the human or animal should thereby be as small as possible, in order to enable it to be passed through as simply and pain-free as possible. On the other hand, the outside diameter should be sufficiently large to give the hollow body sufficient structural stability and/or enable the passage of the required minimum amount of the drug in a predetermined period of time. For this reason, generally peripheral walls with an outside diameter between 0.26 mm and 0.9 mm are preferred, particularly when the delivery device is intended for use for the treatment of virility problems in the region of the penis in humans. For other areas of application, the peripheral wall may, of course, also have an outside diameter differing therefrom, in particular a significantly greater outside diameter. Thus, for example, in the area of veterinary medicine, in particular for the treatment of larger animals, such as for example horses, it is entirely conceivable for the outside diameter of the hollow body to be up to 2 cm in size, or in particular exceptional cases even greater.

In order to prevent unnecessary injuries to the body part of the human or animal to be pierced, it is proposed that the at least one drug outlet opening is rounded or/and provided with a chamfer at least at the transition to the outer surface of the peripheral wall. The smoother this region is, the fewer injuries are to be expected upon on a relative movement of the delivery device to the tissue into which the latter is introduced.

Particularly if the delivery device according to the invention is used, as described above, for the treatment of erectile dysfunction, i.e. is passed through the penis in such a manner that the at least one drug outlet opening is arranged in the region of at least one of the two corpora cavernosa of the penis, the delivery device should on its appropriate use comprise a hollow body with a length at least as great as the width of the erect penis at the place where the delivery device has been passed through the same. At the same time, the length of the hollow body should not be excessively greater, since the protruding sections could otherwise be troublesome. Since the diameter of the erect penis may differ, it can be envisaged to provide the delivery device with hollow bodies of different, predetermined lengths. In this regard, it is proposed that the hollow body has a maximum length of ≦200 mm, preferably of ≦90 mm, more preferably of ≦70 mm, even more preferably of ≦50 mm. Thereby, a length of the hollow body of up to 200 mm is rather in the application area of veterinary medicine of importance. It should be indicated at this point once again that the possible range of application of the delivery device according to the invention is not only limited to the treatment of virility problems.

Alternatively, it is, however, also conceivable to adapt the length of the hollow body of the delivery device individually to the physical circumstances of the patient. To this end, it is conceivable to shorten a respectively long hollow body to the required length by cutting off a section at one or both longitudinal ends of the hollow body. If the hollow body is formed, for example, from a suitably flexible material, it could be envisaged to store it rolled up on a reel and to cut off pieces according to the desired length, as required. When determining the length of the hollow body, it should in any case be ensured that, even on maximal displacement of the hollow body relative to the body part penetrated by the hollow body, an emergence of the at least one drug outlet opening from the body part does not occur, in order to keep the risk of infection low.

It is advantageous, in particular for a long duration in situ of the delivery device in the body of a human or animal, if the hollow body is produced at least in sections, preferably completely, from a biocompatible material. To this end, various materials are to be considered, with experience already being available, particularly from the field of piercing, concerning the long-term tolerability of materials which are processed into objects which penetrate a human body in sections, similar to the delivery device according to the invention. Suitable biocompatible materials may be chosen for example from: niobium, palladium and titanium. Several high-grade steels, which are known colloquially by the term “surgical steel”, such as e.g. X20Cr13 and X5CrMi18-10, may also be used. The use of metallic materials has, inter alia, the advantage that they are particularly easily identifiable in imaging techniques, such as e.g. ultrasound, and this facilitates the positioning when, during the latter, imaging methods are used by medically trained staff. However, also the use of non-metallic materials is perfectly possible, such as e.g. the use of polytetrafluoroethylene, which is better known under the trade name Teflon® of the company Dupont. In addition, there are also various biocompatible plastics which are suitable for the production of the hollow body.

If the delivery device according to the invention is used in the field of animal husbandry and/or veterinary medicine, the present invention furthermore has the advantage that reinjection or repeated introduction of drugs or hormones, respectively, is also possible by staff without special medical training. As a result, considerable costs for repeated injections can be saved, for which a veterinarian and optionally auxiliary staff additionally required, for example to restrain the animal, would otherwise charge. Moreover, the animal to be treated may possibly not have to be anaesthetised several times, thus resulting in a reduction of the anaesthetic supply and in a reduction of the attendant danger of side effects.

In the following, on the one hand a method for arranging a delivery device according to the invention and on the other hand a method for delivering a drug in the tissue of a human or animal using the delivery device according to the invention are described for better understanding of the delivery device according to the invention, and also to describe further aspects of the present invention.

According to a further aspect, the invention relates to a method for arranging the delivery device according to the invention in the tissue of a human or an animal, in order to inject a drug into the tissue, comprising the following step:

a) piercing the tissue with a piercing needle, wherein the hollow body of the delivery device is simultaneously passed through the tissue, so that both longitudinal ends of the hollow body are arranged outside the tissue, whereas a hollow-body section situated between the two longitudinal ends, which preferably has the at least one drug delivery opening, is arranged in the tissue.

As already described above, the piercing needle can be passed through the hollow space of the hollow body, when the hollow body is of tube-like form, the hollow space extending continuously from the first to the second longitudinal end of the hollow body, so that a tip of the piercing needle protrudes from one longitudinal end of the hollow body before the piercing needle together with the hollow body is passed through the tissue of the human or animal.

Since the delivery device according to the invention is designed to dwell permanently, i.e. over months, if not even years, in the body of the human or animal, without further puncturing being necessary during this time, it does not represent an unreasonable burden to carry out the singular arrangement of the delivery device according to the invention by medically trained staff. For an optimal arrangement or positioning, respectively, of the delivery device according to the invention, it is proposed that during the step a) an imaging technique, in particular an ultrasonic imaging technique, is used to ensure the correct arrangement of the hollow body, and in particular of the at least one drug outlet opening, in the tissue.

It is furthermore proposed that the hollow space extends continuously from the first to the second longitudinal end of the hollow body, which is of tube-like form, wherein the piercing needle has been passed through the tube-like hollow body before step a), so that a pointed longitudinal end of the piercing needle protrudes from one longitudinal end of the hollow body.

In such a case, it is furthermore advantageous if the method after step a) furthermore comprises the following step:

b) withdrawing the piercing needle from the hollow body without substantially changing the position of the hollow body relative to the tissue in doing so.

By withdrawing the piercing needle, the lumen surrounded by the peripheral wall of the hollow body is free again and enables the delivery of the drug from an extracorporeal source to the intracorporeally arranged drug outlet opening.

If the tissue is a penis, wherein the arrangement of the delivery device preferably performed in the vicinity of the root of the penis, it is proposed, for the purpose of the above-mentioned individual adaptation of the length of the hollow body to the anatomical circumstances of the patient, that the method after step a), and optionally after step b), furthermore comprises the following steps:

c) causing an erection of the penis, preferably by delivering a drug, which brings about an erection of the penis, through the hollow body penetrating the penis and into the penis; and

d) cutting the hollow body off to a suitable length, so that the two longitudinal ends of the hollow body are arranged at a predetermined distance range from the entry and/or exit point, respectively, of the hollow body.

In order to prevent the hollow body positioned in the human or animal body from slipping out, it is advantageous if the method after step a), and optionally after step b), and preferably optionally after step c) and d), furthermore comprises the following step:

e1) attaching a closure arrangement to one of the two longitudinal ends of the hollow body, in particular if the other of the two longitudinal ends of the hollow body is already provided with a closure arrangement; or

e2) attaching to both longitudinal ends of the hollow body one closure arrangement each.

According to a further aspect, the invention relates to a method for delivering a drug into the tissue of a human or animal using the delivery device according to the invention, which has preferably been arranged in the tissue according to the above-described arranging method, comprising the following step:
delivering the drug from outside the delivery device, via the hollow space and the at least one drug outlet opening, to the tissue.

The delivery method can comprise the following steps:
selecting one of the at least one above-described closure arrangement; and
if this closure arrangement is an above-described, detachable closure arrangement:
detaching the closure arrangement;
delivering the drug, preferably using an injection syringe; and
reclosing the closure arrangement; or
if this closure arrangement has an above-described, reclosable closure means:
opening the closure means;
delivering the drug, preferably using an injection syringe; and
reclosing the closure means; or
if this closure arrangement has an above-described elastic body:
plunging the pointed needle of the injection syringe into or through the stopper;
delivering the drug using the injection syringe; and
withdrawing the needle of the injection syringe from the elastic body.

The invention is explained in more detail below with the aid of several schematic drawings, not to scale, which show different exemplary embodiments.

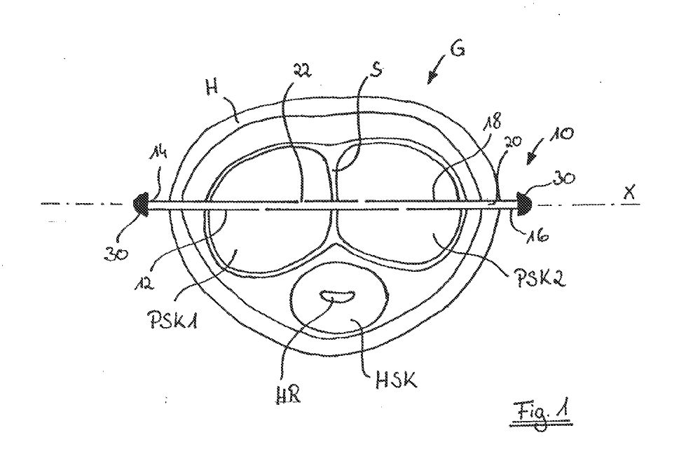

FIG. 1 shows a schematic cross-sectional view, not to scale, of a penis G, wherein the section plane runs substantially orthogonally to the direction of the longitudinal extent of the penis G and in the region of its root. In this schematic cross-sectional view, an outer skin H of the penis G which surrounds two corpora cavernosa PSK1 and PSK2, and a corpus spongiosum HSK are to be seen. The corpus spongiosum HSK for its part surrounds a urethra HR. A septum S is arranged between the two corpora cavernosa PSK1 and PSK2.

One embodiment of the delivery device 10 according to the invention is extending through the penis G. This embodiment comprises, in the embodiment illustrated, an elongate hollow body 12 having a first longitudinal end 14 and a second longitudinal end 16. The hollow body for its part comprises a with respect to the axis of the longitudinal extent X radially outer surrounding wall 18, which wall surrounds a radially inner, elongate hollow space 20. In the present exemplary embodiment, the hollow body 12 is of substantially tube-like form, whereby the elongate hollow space 20 extends continuously from the first longitudinal end 14 to the second longitudinal end 16. Between the two longitudinal ends 14 and 16 of the hollow body 12, the peripheral wall 18 in this exemplary embodiment has a plurality of drug outlet openings 22. These drug outlet openings are preferably arranged spaced apart from one another along the direction of the longitudinal extent X of the hollow body 12 of the delivery device 10, on the one hand in order not to excessively weaken the structural integrity of the hollow body locally, and on the other hand in order to achieve for a better distribution of the drug in the tissue, i.e. in the present case in the corpora cavernosa PSK1 and PSK2. In the exemplary embodiment shown, the peripheral wall 18 of the hollow body 12 has four drug outlet openings 22, which are arranged in such a manner that, in the state when the drug delivery device 10 is positioned in the penis, two drug outlet openings are positioned in the first corpus cavernosum PSK1, whereas the other two drug outlet openings 22 are arranged in the second corpus cavernosum PSK2.

It should be noted that, to achieve the desired effect on appropriate use of the drug delivery device illustrated here, namely to cause an erection of the penis, in principle a single drug outlet opening 22 in one of the two corpora cavernosa PSK1 and PSK2 would be sufficient, since the drug would spread by itself in both corpora cavernosa PSK1 and PSK2 starting from the one drug outlet opening 22. However, the provision of a plurality of drug outlet openings 22 has the advantage that the distribution process is accelerated and thus the desired effect can be brought about more quickly. Furthermore, the provision of a plurality of drug outlet openings 22 has the advantage that the safeguarding against failure of the drug delivery device according to the invention can be increased, since the device 10 is still able to work even if, over time, one or more drug outlet openings 22 have become clogged, as long as at least one drug outlet opening 22 is still accessible starting from one of the two longitudinal ends 14, 16 of the hollow body 12. In view of the fact that the drug delivery device 10 according to the invention is intended in particular for permanent use, i.e. for use over several months, if not even years or decades, a high reliability is of increased importance.

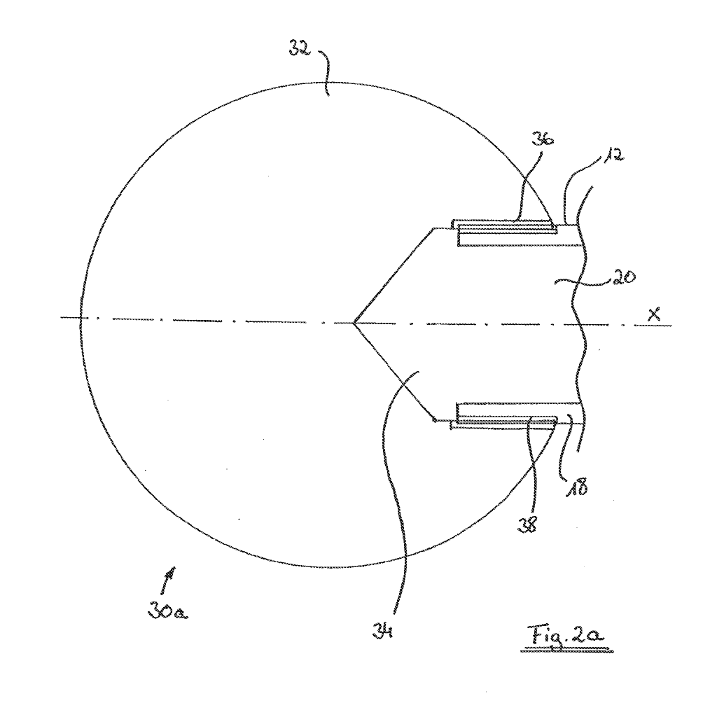

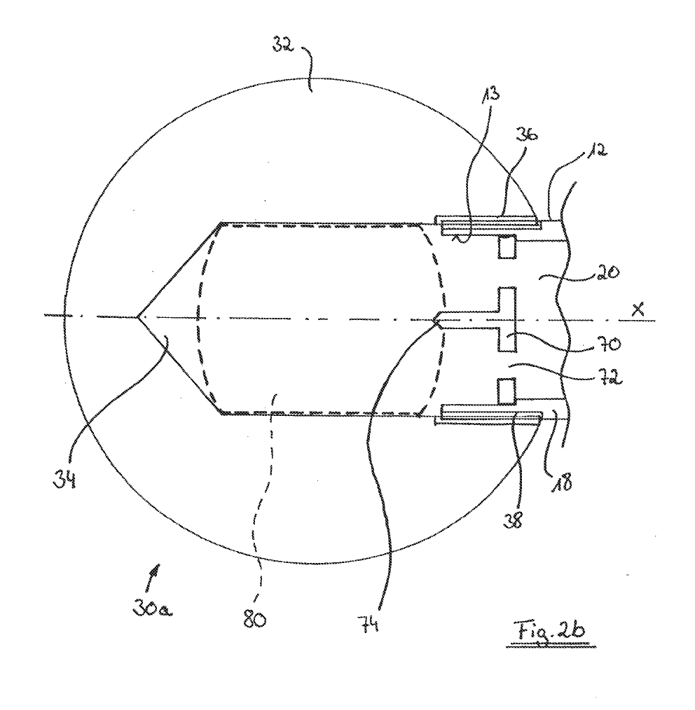

Furthermore, the delivery device 10 illustrated in FIG. 1 comprises, at both longitudinal ends 14, 16 of the hollow body 12, a closure arrangement 30 each, illustrated here uttermost schematically only, whereby different embodiments of the closure arrangement 30 are explained in more detail below with regard to FIGS. 2 to 6. The two closure arrangements 30 are arranged at the two longitudinal ends 14, 16 of the hollow body 12 in such a manner that they can prevent germs, bacteria or other pathogens from penetrating from outside of the delivery device 10 into the hollow space 20, and thus into the tissue surrounded by the skin H of the penis G. At the same time, the closure arrangements 30 are, as will be explained in more detail below, configured in such a manner that they allow a drug, in the present application in particular a liquid impotence drug, to be fed from an extracorporeally situated drug source into the hollow space 20 of the device 10. For example, a syringe provided with an injection needle can be used to introduce the drug into the hollow space 20 of the delivery device 10, from where the drug is then guided to the drug outlet openings 22 and from there the drug can penetrate into the tissue, i.e. in the present application into the two corpora cavernosa PSK1 and PSK2.

As likewise merely schematically indicated in FIG. 1, the two closure arrangements 30 have a diameter, i.e. a radial dimension with respect to the axis of the longitudinal extent X of the hollow body 12 of the delivery device 10, which is significantly greater than the corresponding radial dimension of the hollow body 12. The hollow body 12, and/or its peripheral wall 18, respectively, is in the present case substantially formed in the shape of a straight circular hollow cylinder. The positioning of the hollow body 12 in the tissue, i.e. in the present case in the penis G, is preferably carried out in a state in which at least one, preferably both, closure arrangements 30 are not yet connected to the hollow body 12. For example, if neither of the two closure devices 30 is yet connected to the hollow body 12, a long, pointed piercing needle can be passed through the elongate hollow space 20 of the hollow body 12, so that the tip of the piercing needle protrudes from one of the two longitudinal ends 14, 16 of the hollow body 12. Subsequently, the piercing needle, together with the hollow body 12, can be plunged through the tissue, i.e. in the present case the penis G, optionally using a suitable aid, thereby positioning the hollow body 12 in the tissue in such a manner that its two longitudinal ends 14, 16 protrude from the tissue, while a section arranged between the two longitudinal ends 14, 16 which has the at least one drug outlet opening 22, is arranged inside the tissue. Subsequently, the piercing needle can be withdrawn from the hollow body in order to free the hollow space 20 again. In doing so, care has to be taken that the relative position of the hollow body 12 in relation to the tissue is substantially not changed. Subsequently, at the two longitudinal ends 14, 16 of the hollow body, one closure arrangement 30 can each be arranged. Since the closure arrangements 30 have a greater radial dimension than the hollow body 12 as described above, they prevent the hollow body 12 from unintentionally slipping out of the tissue, i.e. in the present case the penis G. This provision has a great advantage for the permanent use of the delivery device according to the invention, since in other, similar devices, such as e.g. so-called indwelling venous catheters, there is always the risk of slipping out, in particular when the person concerned moves a lot. In contrast, the delivery device 10 according to the invention can be reliably kept permanently in the tissue to which a drug is to be delivered, similar to a genital piercing.

Particularly for use of the drug delivery device 10 according to the invention in the field of application illustrated in FIG. 1, it should be noted that the diameter of the penis G can vary according to the person concerned. It is therefore possible to use either drug delivery devices with hollow bodies of different standard sizes, or it can be envisaged to adapt the length of the hollow body 12 individually according to the anatomical circumstances. The latter can be done either before the hollow body 12 has even been positioned in the penis G, or else the adaptation can also be done only after the positioning, preferably by medically trained staff who carry out the positioning.

For example, it is possible for the hollow body 12 to have a length in the axial direction X which is significantly greater than the required length, which is dimensioned according to the diameter of the penis G at the corresponding place in the aroused state. In such a case, it is possible to shorten the hollow body 12 to the required length either on one side or on both sides after the positioning in the penis G. Particularly if the hollow body 12 is formed substantially from a metallic material, this hollow body can be readily visualised by conventional imaging techniques, such as, for example, by ultrasound. Such an imaging method can be used, during the positioning of the hollow body 12 in the penis G, in particular to ensure that the at least one drug outlet opening 22 is or are optimally positioned with respect to the corpora cavernosa PSK1 and PSK2. The at least one drug outlet opening 22 should be spaced from the longitudinal ends 14, 16 of the hollow body 12 such that, even on a maximum displacement along the direction of the extent X of the hollow body 12 relative to the penis in the flaccid state, the at least one drug outlet opening 22 is reliably prevented from leaving the penis G, preferably also formulating the respective corpus cavernosum PSK1 and PSK2, respectively, in order to avoid penetration of germs or the like via the at least one drug outlet opening 22.

The schematic cross-sectional view illustrated in FIG. 1 shows the penis G in a moderately erect state, in which the closure arrangements arranged at the two longitudinal ends 14, 16 of the hollow body 12 are still at a marked distance from the skin H of the penis G. In an extremely erect state of the penis G, these closure arrangements can also rest against the skin H, but they should substantially not exert any pressure on the penis G. A significantly greater dimension in the longitudinal direction X of the hollow body 12 is, however, not advantageous, since in such case the delivery device 10 could be obstructive. As already mentioned, the delivery device is preferably arranged in the region of the root of the penis G, in order not to be troublesome during sexual intercourse. Particularly if the hollow body 12 is produced substantially from a metallic material, it is advantageous if the dimensions of the hollow body 12 in the radial direction are relatively small, i.e. preferably ≦0.9 mm, so that the hollow body 12 can be cut to length in a simple manner, for example by means of pliers, similar to a projecting wire of dental braces. However, in order to ensure sufficient stability of the device on the one hand and a satisfactory flow rate per unit of time on the other hand, the outside diameter of the hollow body should be at least 0.26 mm. The hollow body 12 of the delivery device 10 should preferably be produced from a biocompatible material suitable for permanent use in the corresponding tissue. To this end, in particular materials such as those known, for example, from the field of piercing, are possible.

FIG. 2a shows a schematic cross-sectional view of a first embodiment of a closure arrangement 30a arranged at a longitudinal end of the hollow body 12. The closure arrangement 30a comprises a sphere-like closure body 32, in which a blind hole 34 is provided. The blind hole 34 comprises an internal-thread section 36 which is formed complementarily to an external-thread section 38 provided at the corresponding longitudinal end of the hollow body 12. In this way, the closure body 32 can be repeatedly connected to and/or detached from the hollow body 12 in a simple manner, in order to introduce a drug into the hollow space 20 of the hollow body 12 and to additionally prevent pathogens from penetrating into the hollow space 20, respectively. When delivering the drug into the hollow space 20, naturally care must be taken that the opposite longitudinal end (not illustrated in FIG. 2a) of the hollow body 12 is closed, in order to prevent the drug from leaking from this longitudinal end and thus to ensure that the drug arrives in the tissue to be treated, via the at least one drug outlet opening 22. The round shape of the sphere-like closure body 32 reduces the risk of injury and the risk that the person wearing the delivery device 10 has an unpleasant feeling, respectively. Furthermore, it is conceivable to design the closure body 32 similar to a piece of jewellery, so that the delivery device 10 according to the invention resembles a piercing.

Since it is necessary, in the embodiment of the closure arrangement 30a illustrated in FIG. 2a, for the corresponding longitudinal end of the hollow body 12 to be provided with an external-thread section 38, it is advantageous to provide this embodiment 30a of the closure arrangement only on one side, in order to prevent the longitudinal end of the hollow body 12 having the external-thread section 38 from having to be passed through the tissue, in particular the penis G, which otherwise could possibly result in increased impairment of the penetrated tissue due to the surface unevenness of the external-thread section 38. Although, in principle, it would be conceivable to produce the external-thread section 38 only after the arrangement of the hollow body 12 in the tissue, this is usually relatively complex and costly.

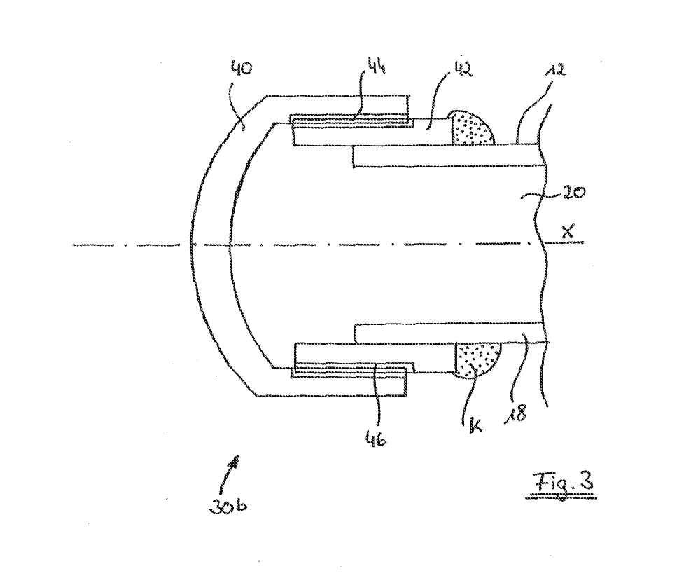

A solution to this is provided, for example, by the second embodiment of a closure arrangement 30b illustrated in FIG. 3. The closure arrangement 30b further comprises, in addition to a closure element 40 of cap-like form, a hollow-cylinder-shaped sleeve element 42, the inside diameter of which corresponds substantially to the outside diameter of the peripheral wall 18 at the corresponding longitudinal end of the hollow body 12, so that the sleeve element 42 can be pushed over the longitudinal end of the hollow body 12, at least in sections. The sleeve element 42 can then be permanently connected to the peripheral wall 18 of the hollow body 12, preferably by means of an adhesive bonding K. The sleeve element 42 can be connected to the corresponding longitudinal end of the hollow body 12 in particular only after this end has been passed through the tissue, in particular the penis G.

Similar to the embodiment described in FIG. 2a, the closure element 40 has an internal-thread section 44 which is formed complementarily to an external-thread section 46 provided at the sleeve element 42. Thus, the closure element 40 can be repeatedly connected to the sleeve element 42—and thus to the hollow body 12—or detached therefrom again, respectively, in order to enable the delivery of a drug into the hollow space 20 of the hollow body 12, and, in order to otherwise reliably prevent the penetration of pathogens. In principle, it should be noted that additionally also sealing means, such as for example a sealing ring or the like, may be provided between the sleeve element 42 and the closure element 40.

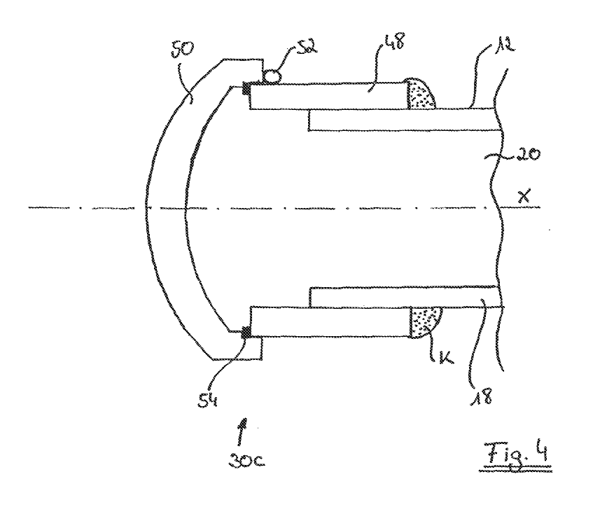

The two embodiments 30a, 30b of the closure arrangement described above stand out in that the respective closure element 32 and 40, respectively, is completely separable from the hollow body 12. However, this involves the risk that the closure element 32, 40, which is detached from the hollow body during the delivery of the drug, can be lost, or accidently dropped and thereby contaminated. In order to prevent this, it is proposed, in a third embodiment of a closure arrangement 30c illustrated in FIG. 4, to connect a respective closure element 50 permanently to a corresponding sleeve element 48 by means of a hinge device 52. Thus, the closure element 50 can be opened and closed like a cap, and, in doing so, cannot go missing. The sleeve element 48 illustrated in the embodiment 30c corresponds substantially to the sleeve element 42 illustrated in FIG. 3, but it has no external-thread section. Likewise, the closure element 50 of FIG. 4 has no internal-thread section. Otherwise, however, reference is made to the embodiment according to FIG. 3. The joint or hinge 52 can be designed, for example, as a film hinge, in particular if the sleeve element 48 and the closure element 50 are produced from plastic. Thereby, the sleeve element 48, the closure element 50 and the articulation 52 can be integrally produced. The embodiment of the closure arrangement 30c illustrated in FIG. 4 furthermore has a ring-shaped sealing element 54 which reliably prevents the penetration of germs or other pathogens into the hollow space 20 of the hollow body 12 when the closure element 50 of cap-like form is closed.

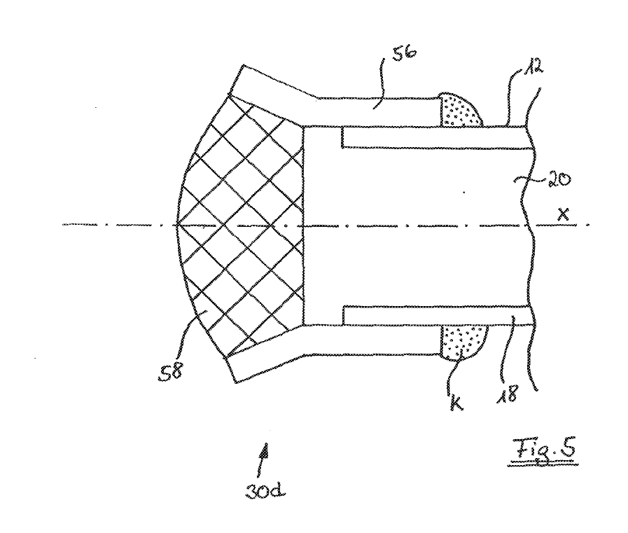

A fourth embodiment of a closure arrangement 30d is illustrated in FIG. 5. In this embodiment, a sleeve element 56, which can be connected to the peripheral wall 18 of the hollow body 12, as in the two embodiments described above, widens in the shape of a funnel towards its free longitudinal end. The funnel-shaped section of the sleeve element 56 is blocked herein by an elastic closure element 58, preferably in the shape of a rubber plug. The rubber plug prevents the penetration of germs or other pathogens into the hollow space 20 of the hollow body 12. At the same time, the rubber plug 58 allows to be penetrated into it or through it by a pointed cannula of an injection syringe, by means of which a drug can be injected into the hollow space 20 of the hollow body 12. Due to the elastic properties of the closure element 58, the latter closes again by itself as soon as the cannula is withdrawn. The handling of such a closure arrangement 30d is thus very simple. Moreover, the section of the sleeve element 56 which widens in the shape of a funnel facilitates the insertion of the cannula, since the area into which it can be inserted is markedly greater than the cross-sectional area—orthogonal to the direction of the longitudinal extent X—of the hollow space 20 of the hollow body 12.

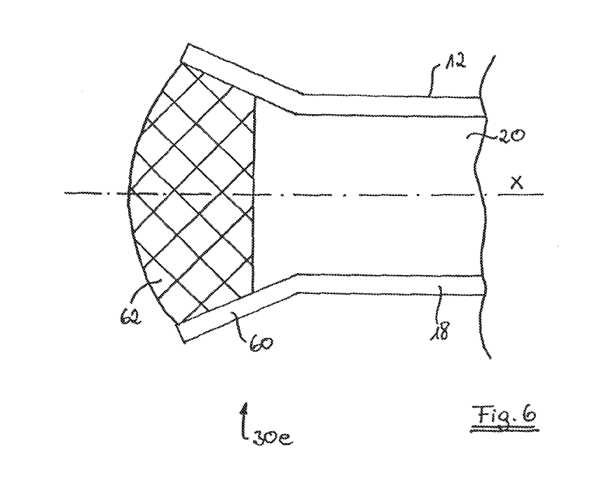

FIG. 6 shows a further embodiment of a closure arrangement 30e which is similar to that of FIG. 5. In the embodiment 30e illustrated in FIG. 6, however, the section widening in the shape of a funnel is integrally formed with the peripheral wall 18 of the hollow body 12. As in the embodiment described above, this section widening in the shape of a funnel is also closed by means of an elastic closure element 62 which can be formed as an elastic rubber stopper. However, a delivery device 10 can have a closure arrangement 30e, as illustrated in FIG. 6, only at one of the two longitudinal ends 14, 16 of the hollow body 12, since this closure arrangement is not suitable to be passed through the tissue, in particular a penis G, due to its large radial diameter. At the opposite longitudinal end of the hollow body 12 can, after the positioning of the hollow body 12 in the tissue, for example one of the closure arrangements 30b, 30c, 30d illustrated in FIGS. 3 to 5.

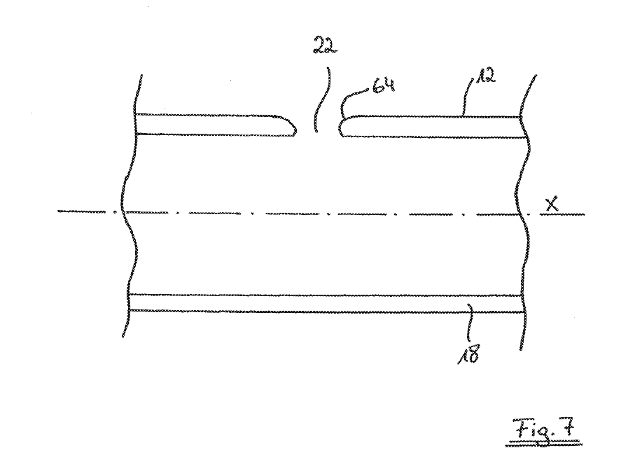

FIG. 7 shows a schematic, likewise greatly enlarged cross-sectional view of a section which is situated between the two longitudinal ends 14, 16 of the hollow body and has a drug outlet opening 22. The drug outlet opening 22 passes through the peripheral wall 18 of the hollow body 12 in substantially radial direction. As can be seen clearly in FIG. 7, the transition region 64 of the drug outlet opening 22 is rounded off towards the outer surface of the peripheral wall 18 of the hollow body 12, i.e. it is free from pointed corners and edges. Alternatively, the peripheral wall 18 could also have a chamfer at this place. This has the advantage that no or fewer injuries occur when passing the hollow body 12 through the tissue, for example of a penis of a human or animal to be treated.

The delivery device 10 according to the invention stands out in particular in that it is suitable for permanent and long-term use, respectively, so that in cases in which a delivery of drugs is necessary repeatedly at more or less regular intervals, the skin does not have to be punctured again and again in order to guide the drug from an extracorporeal source to an intracorporeal destination.

In FIG. 2b, a variant of the first embodiment illustrated in FIG. 2a is shown. Functionally identical or similar components or component sections have the same reference signs in FIG. 2b as in FIG. 2a; reference is therefore made to the above description relating to FIG. 2a with regard to these components or component sections.

In the variant according to FIG. 2b, the substantially spherically shaped closure body 32 of the closure arrangement 30a has a blind hole 34 which is longer than the blind hole 34 of the first embodiment illustrated in FIG. 2a and is thus suitable for serving as a receptacle 34 for a drug reservoir 80 (indicated merely by dashed lines in FIG. 2b). The drug reservoir 80 may be, for example, a substantially elastically formed envelope (similar to a balloon) which is filled with the drug to be applied, for example an impotence drug or else a corresponding antidote. Thereby, the drug reservoir 80 can have, for example, the basic shape of a sphere which adapts to the cylindrical contour of the receptacle 34 on introduction into the drug reservoir receptacle 34, or the drug reservoir 80 can already have a substantially cylindrical basic shape which is matched to the contour of the receptacle 34. In the latter case, the elasticity of the drug reservoir 80 is only of minor importance.

Furthermore, the variant according to FIG. 2b differs from the first embodiment according to FIG. 2a in that a groove 13 is provided at that the illustrated longitudinal end of the hollow body 12 radially inside. A plate 70 is inserted into the groove 13 and preferably fixedly connected to the hollow body 12, for example by adhesive bonding. The plate 70 has at least one through-hole 72. For example, the plate 70 can have four through-holes 72 distributed uniformly in an annular way around a centre point of the substantially circular plate 70. The through-holes 72 serve to let the drug to be applied pass from an extracorporeal source, namely the drug reservoir 80, to the drug outlet openings 22 provided in the hollow body 12. Moreover, the plate 70 comprises a pointed or sharp-edged projection 74. In the present exemplary embodiment, this pointed projection 74 is formed in the shape of a spike which extends from the centre of the plate 70 towards the drug reservoir 80. It should be noted, however, that the pointed or sharp-edged projection could also be differently formed, e.g. integral with the hollow body 12. All that is important is that the projection extends towards the drug reservoir 80 in order to achieve the following, desired effect:

If the drug reservoir 80 is inserted into the drug reservoir receptacle 80, which is provided in the closure body 32, and if the closure body 32 is then connected to the hollow body 12, for example by screwing on, the pointed or sharp-edged projection 74 comes into contact with the drug reservoir 80 and destroys the latter in a targeted manner. Thus, the precisely predetermined amount of drug contained in the drug reservoir 80 can leak from the latter and pass through the through-holes 72 in the plate 70, the hollow space 20 of the hollow body 12 and the drug outlet openings 22 provided in the hollow body 12 to the corpora cavernosa PSK1 and PSK2 of the penis G and disseminate the desired effect there. Thereby, the plate 70 prevents the destroyed envelope of the emptied drug reservoir 80 from getting likewise into the hollow space 20 of the hollow body 12. Rather, the envelope remains in the receptacle 34 of the closure body 32 and can be easily removed by the user and, if required, replaced by an intact drug reservoir 80.

It should be noted that a drug reservoir receptacle and a pointed and sharp-edged, respectively, projection can also be provided in a similar manner in the above-described devices according to FIGS. 3 and 4.

Furthermore, it should be noted that the pointed or sharp-edged projection can also be dispensed with, namely for example if the drug reservoir has an integrated predetermined point of rupture and if the closure body is formed from an elastic or flexible material. In this case, a firm pressure on the closure body containing the drug reservoir is sufficient to destroy the drug reservoir in a targeted manner so that the drug can come out. To this end, the closure body can be formed, for example, similar to the proximal end of a typical pipette for nose drops.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C

1. A device for delivering a drug, the device comprising:
an elongate hollow body having a first longitudinal end and a second longitudinal end, the hollow body comprising a radially outer peripheral wall, which surrounds a radially inner, elongate hollow space, wherein the peripheral wall of the hollow body has at least one drug outlet opening between the two longitudinal ends.

2. The device according to claim 1, wherein the hollow body is of tube-like form, the hollow space extending continuously from the first longitudinal end to the second longitudinal end of the hollow body.

3. The device according to claim 1, wherein the hollow body has a plurality of drug outlet openings.

4. The device according to claim 3, wherein the drug outlet openings are arranged spaced apart from one another with respect to the direction of a longitudinal extent of the hollow body.

5. The device according to claim 1, wherein the device furthermore comprises at least one closure arrangement which is attachable to the respective longitudinal end of the hollow body in order to sealingly close the hollow space at the respective longitudinal end of the hollow body.

6. The device according to claim 5, wherein at least one of the at least one closure arrangement is configured to enable the delivery of a drug from outside the delivery device into the hollow space.

7. The device according to claim 5, wherein at least one of the at least one closure arrangement is detachably connected or connectable, via a screwed connection, to the hollow body.

8. The device according to claim 5, wherein at least one of the at least one closure arrangement is non-detachably connected, via an adhesive bonded connection, to the hollow body.

9. The device according to claim 5, wherein at least one of the at least one closure arrangement comprises a closure mechanism configured for opening and reclosing.

10. The device according to claim 5, wherein at least one of the at least one closure arrangement comprises an elastic body, which is configured to be pierced with a pointed needle, in order to introduce a drug into an interior of the hollow body, and which is configured, on account of its elastic properties, for sealingly reclosing the hollow space at the corresponding longitudinal end of the hollow body when the needle is withdrawn.

11. The device according to claim 5, wherein at least one of the at least one closure arrangement extends in the radial direction of the hollow body further radially outwards than the hollow body.

12. The device according to claim 1, wherein the peripheral wall has a minimum outside diameter in the radial direction, which is at least about 0.26 mm.

13. The device according to claim 1, wherein at least one drug outlet opening is at least one of rounded off at least at a transition to the outer surface of the peripheral wall or provided with a chamfer.

14. The device according to claim 1, wherein the hollow body has a length of at most about 200 mm.

15. The device according to claim 1, wherein the hollow body is produced at least sectionally from a biocompatible material.

16. The device according to claim 1, wherein the device furthermore comprises a sharp-edged projection configured to destroy an envelope of a drug reservoir in a targeted local manner, so that an amount of a drug can leak from the reservoir into an interior of the hollow body, whereby the sharp-edged projection is concealed while access to the interior of the hollow body is closed by a closure arrangement at one of the longitudinal ends of the hollow body.

17. The device according to claim 1, wherein the device has at least one of a drug reservoir or a receptacle for a drug reservoir, whereby the drug reservoir or the receptacle for the drug reservoir is disposed at least one of in or at a closure arrangement of the device.

18. A method for arranging a delivery device in tissue of a human or an animal, in order to inject a drug into the tissue, the device including an elongate hollow body having a first longitudinal end and a second longitudinal end, the hollow body comprising a radially outer peripheral wall, which surrounds a radially inner, elongate hollow space, wherein the peripheral wall of the hollow body has at least one drug outlet opening between the two longitudinal ends, the method comprising:
piercing the tissue with a piercing needle, whereby the hollow body of the device is simultaneously passed through the tissue, so that both longitudinal ends of the hollow body are disposed outside the tissue, whereas a hollow-body section situated between the two longitudinal ends and having the at least one drug delivery opening is disposed in the tissue.

19. The method according to claim 18, wherein an imaging technique, in particular an ultrasonic imaging technique, is used to ensure the correct arrangement of the hollow body (12), and in particular of the at least one drug outlet opening (22), in the tissue.

20. The method according to claim 18, wherein the hollow space extends continuously from the first to the second longitudinal end of the hollow body, which is of tube-like form, whereby the piercing needle has been passed through the tube-like hollow body prior to piercing the tissue with the piercing needle, so that a pointed longitudinal end of the piercing needle protrudes at one longitudinal end of the hollow body.

21. The method according to claim 20, wherein the method furthermore comprises withdrawing the piercing needle from the hollow body without substantially changing the position of the hollow body relative to the tissue.

22. The method according to claim 18, wherein the tissue is a penis and the method furthermore comprises:
causing an erection of the penis by delivering a drug, which brings about an erection of the penis, through the hollow body penetrating the penis and into the penis; and
cutting the hollow body to a suitable length, so that the two longitudinal ends of the hollow body are disposed at a predetermined distance range from the entry and exit point, respectively, of the hollow body with respect to the penis.

23. The method according to claim 18, wherein the method furthermore comprises at least one of attaching a closure arrangement to one of the two longitudinal ends of the hollow body or attaching to both longitudinal ends of the hollow body one closure arrangement each.

24. The method according to claim 18, further comprising:
delivering the drug from outside the delivery device, via the hollow space and the at least one drug outlet opening, to the tissue.

25. The method according to claim 24, wherein the device further comprises at least one closure arrangement which is attachable to the respective longitudinal end of the hollow body in order to sealingly close the hollow space at the respective longitudinal end of the hollow body, wherein the method further comprises at least one of:
detaching the closure arrangement, delivering the drug using an injection syringe, and reclosing the closure arrangement; or
opening a closure mechanism, delivering the drug using an injection syringe, and reclosing the closure mechanism; or
plunging a pointed needle of an injection syringe through an elastic body, delivering the drug using the injection syringe, and withdrawing the needle of the injection syringe from the elastic body.
